(19) United States
(12) Patent Application Publication
(10) Pub. No.: US 2004/0215181 A1
(43) Pub. Date: Oct. 28, 2004

(54) Delivery of fluid during transurethral prostate treatment

(75) Inventors: Mark A. Christopherson, Shoreview, MN (US); Thomas Richard Skwarek, Shoreview, MN (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc., Minneapolis, MN

(21) Appl. No.: 10/424,040

(22) Filed: Apr. 25, 2003

(51) Int. Cl. A61B 18/18

(52) U.S. Cl. 606/32; 606/41

(57) ABSTRACT

In general, the invention provides a transurethral ablation device comprising a fluid delivery port for delivery of a fluid, such as a gel, liquid, or liquid suspension, to a tissue site targeted for ablation. The fluid is electrically conductive and carries a local anesthetic agent. When the fluid is delivered, it provides an anesthetic effect in the target tissue site, alleviating pain associated with the ablation procedure. In addition, the fluid is loaded with electrically conductive material to enhance conductivity and permit the creation of a virtual electrode. The virtual electrode extends throughout the volume of fluid delivered to the target tissue site, and enhances the volumetric coverage and precision of the ablation procedure with the prostate tissue.

### FIELD OF THE INVENTION

The invention relates generally to prostate treatment and, more particularly, to techniques for transurethral treatment of benign prostatic hypertrophy (BPH).

### BACKGROUND

Benign prostatic hypertrophy or hyperplasia (BPH) is one of the most common medical problems experienced by men over 50 years old. Urinary tract obstruction due to prostatic hyperplasia has been recognized since the earliest days of medicine. Hyperplastic enlargement of the prostate gland often leads to compression of the urethra, resulting in obstruction of the urinary tract and the subsequent development of symptoms including frequent urination, decrease in urinary flow, nocturia, pain, discomfort, and dribbling.

One surgical procedure for treating BPH is transurethral needle ablation (TUNA). The TUNA technique involves transurethral delivery of an electrically conductive needle to the prostate site. The needle penetrates the prostate in a direction generally perpendicular to the urethral wall, and delivers electrical current to ablate prostate tissue. The electrical current heats tissue surrounding the needle tip to destroy prostate cells, and thereby create a lesion within the prostate gland. The destroyed cells may be absorbed by the body, infiltrated with scar tissue or become non-functional.

Other transurethral ablation procedures involve delivery of microwave, radio frequency, acoustic, and light energy to the prostate gland. In addition, some procedures involve delivery of localized chemotherapy, drug infusions, collagen injections, or injections of agents which are then activated by light, heat or chemicals to destroy prostate tissue. These procedures, as well as the TUNA procedure, involve tissue trauma that can be painful for the patient. Accordingly, minimization of patient pain continues to be an objective in the design and delivery of transurethral prostate treatment procedures.

In addition, many transurethral ablation procedures lack a desired degree of precision. For example, the precision and uniformity of the procedures in terms of the ability to target specific prostate tissue raises challenges. Also, ablation of specific shapes and volumes of prostate tissue continues to be difficult.

U.S. Pat. No. 6,551,300 to McGaffigan discloses a transurethral ablation device that delivers a topically applied anesthetic agent gel to a urethral wall. U.S. Published Patent Application no. 2002/0183740 to Edwards et al. discloses a transurethral ablation device to ablate prostate tissue via electrically conductive needles. U.S. Pat. No. 6,241,702 to Lundquist et al. describes another transurethral ablation needle device. U.S. Pat. No. 6,231,591 describes instruments for localized delivery of fluids to a portion of body tissue, including the prostate. U.S. Pat. No. 6,537,272 to Christopherson et al. describes creation of a virtual electrode by delivery of a conductive fluid to a tissue site. Table 1 below lists documents that disclose devices for transurethral ablation of prostate tissue.

TABLE 1

| Patent Number | Inventors | Title |
|---|---|---|
| 2002/0183740 | Edwards et al. | Medical probe device and method |
| 6,551,300 | McGaffigan | Device and method for delivery of |
| | | topically applied local anesthetic to wall |
| | | forming a passage in tissue |
| 6,241,702 | Lundquist et al. | Radio frequency ablation device for |
| | | treatment of the prostate |
| 6,231,591 | Desai | Method of localized fluid therapy |
| 6,537,272 | Christopherson | Apparatus and method for creating, |
| | et al. | maintaining, and controlling a virtual |
| | | electrode used for the ablation of tissue |

All documents listed in Table 1 above are hereby incorporated by reference herein in their respective entireties. As those of ordinary skill in the art will appreciate readily upon reading the Summary of the Invention, Detailed Description of the Preferred Embodiments and Claims set forth below, many of the devices and methods disclosed in the patents of Table 1 may be modified advantageously by using the techniques of the present invention.

### SUMMARY

The present invention is directed to a device and method for delivery of fluid during transurethral prostate treatment, e.g., transurethral ablation of prostate tissue to alleviate BPH. The invention has certain objects. That is, various embodiments of the present invention provide solutions to one or more problems existing in the prior art with respect to the ablation of prostate tissue.

The problems include, for example, pain associated with some existing transurethral ablation techniques. In existing techniques, such as the TUNA procedure, electrode needles are deployed into the urethral wall to penetrate prostate tissue to be ablated. The needles deliver energy to ablate prostate tissue and thereby form lesions. Penetration of the needles and delivery of ablation energy can be painful for the patient. Delivery of general anesthetic significantly complicates the surgical procedure, takes time for administration, and affects the patient's overall system and well being.

Another problem concerns the precision of existing transurethral ablation techniques. For example, the precision and uniformity of the procedures in terms of the ability to target specific prostate tissue raises challenges. Also, ablation of specific shapes and volumes of prostate tissue continues to be difficult. In addition, transurethral ablation techniques can result in ablation of insufficient or irregular volumes of prostate tissue.

Various embodiments of the present invention have the object of solving at least one of the foregoing problems. For example, it is an object of the present invention to overcome at least some of the disadvantages of the foregoing procedures by providing a mode for pain relief that involves a localized anesthetic effect. In other words, the invention provides a transurethral ablation procedure that achieves localized pain prevention or relief during the course of the ablation procedure. As another object, the invention provides an a transurethral ablation procedure that provides greater control and precision in the ablation of selected regions of prostate tissue.

Various embodiments of the invention may possess one or more features capable of fulfilling the above objects. In general, the invention provides a transurethral ablation device comprising a fluid delivery port for delivery of a fluid, such as a gel, liquid, or liquid suspension, to a tissue site targeted for ablation. The fluid carries a local anesthetic agent. When the fluid is delivered, it provides an anesthetic effect in the target tissue site, alleviating pain associated with the ablation procedure. In addition, the fluid is electrically conductive to enhance conductivity and permit the creation of a virtual electrode. The virtual electrode extends throughout the volume of fluid delivered to the target tissue site, and enhances the volumetric coverage and precision of the ablation procedure with the prostate tissue.

The invention also provides a transurethral ablation procedure embodied by a method for use of the ablation device described above. The method involves, for example, inserting a distal end of a catheter into a urethra of a male patient, delivering a fluid to a target tissue site, deploying an ablation probe, and applying ablation energy. In this manner, the fluid can be delivered to reduce pain associated with application of the ablation energy. In addition, the fluid creates a virtual electrode for greater volumetric coverage and precision in the ablation procedure. In some embodiments, the fluid may be delivered before, during and after delivery of ablation energy. Also, the fluid may be applied before the ablation probe is deployed so that pain associated with penetration of the urethral wall can also be reduced.

As a further feature, the fluid delivered via the transurethral ablation catheter may include a steroid to promote healing of prostate tissue following the ablation procedure. The steroid may be mixed with the conductive fluid, and may be mixed with the anesthetic agent in the fluid. In this case, the steroid may be delivered before, during or after the ablation procedure. Alternatively, the steroid may be delivered independently of the conductive/anesthetic fluid. For example, the steroid may be delivered following the ablation procedure. In addition, the fluid may carry a vaso-constrictor that serves to constrict blood vessels in the ablation zone. In this manner, the vaso-constrictor tends to reduce blood flow that otherwise would contribute to cooling in the ablation zone, and thereby reduce the concentration of ablation energy and prolong the time needed for effective ablation.

In comparison to known implementations of transurethral prostate ablation, various embodiments of the present invention may provide one or more advantages. In general, the invention may reduce the pain associated with some existing transurethral ablation techniques. Also, the invention offers a localized treatment for alleviation of pain. In addition, in some embodiments, the fluid can be delivered by the same device used to perform the transurethral ablation procedure, making the procedure less complex, quicker, and more convenient for the surgeon. As a further advantage, the invention provides greater volumetric coverage and precision in the ablation procedure, enabling a greater volume of prostate tissue to be more uniformly ablated within a given ablation procedure.

The above summary of the present invention is not intended to describe each embodiment or every embodiment of the present invention or each and every feature of the invention. Advantages and attainments, together with a more complete understanding of the invention, will become apparent and appreciated by referring to the following detailed description and claims taken in conjunction with the accompanying drawings.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a schematic diagram illustrating a device for transurethral ablation of prostate tissue in accordance with the invention.

FIG. 2 is an enlarged view of the distal end of the device of FIG. 1.

FIG. 3 is another enlarged view of the distal end of the device of FIG. 1.

FIG. 4 is a side view of an ablation needle equipped to deliver a fluid to a target tissue site.

FIG. 5 is a side view of an alternative ablation needle equipped to deliver a fluid to a target tissue site.

FIG. 6 is a side view of another alternative ablation needle equipped to deliver a fluid to a target tissue site.

FIG. 7 a side view of a further alternative ablation needle equipped to deliver a fluid to a target tissue site.

FIG. 8 is a side view of an ablation catheter incorporating multiple ablation needles for delivery of a fluid.

FIG. 9 is a flow diagram illustrating a transurethral ablation procedure in accordance with the invention.

FIG. 10 is a flow diagram illustrating another transurethral ablation procedure in accordance with the invention.

### DETAILED DESCRIPTION OF THE PREFERRED EMBODIMENTS

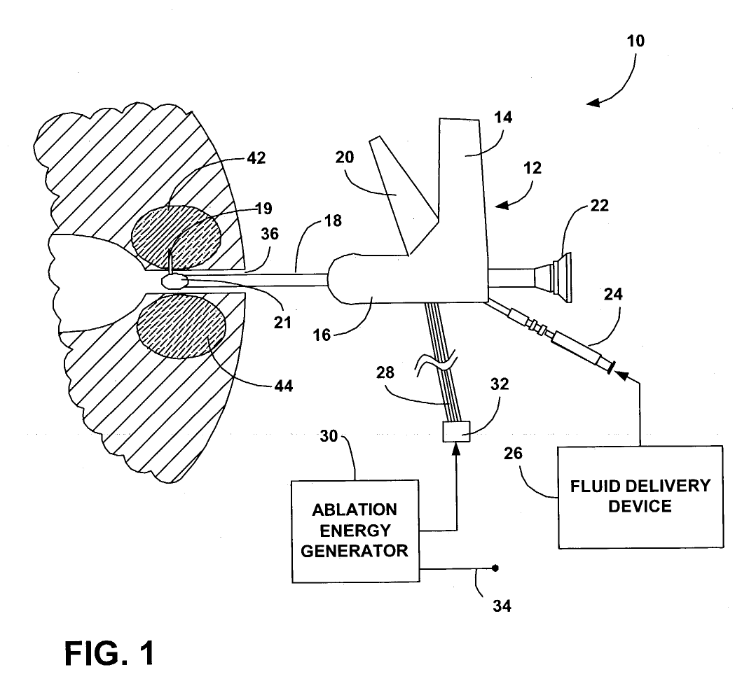

FIG. 1 is a schematic diagram illustrating a device 10 for transurethral ablation of prostate tissue. In accordance with the invention, device 10 is capable of delivering a fluid to a patient to prevent or relieve pain associated with the transurethral ablation procedure, and to create a virtual electrode for more effective and precise ablation. To that end, the fluid may include an anesthetic agent for pain relief and conductive particles, such as conductive salts, to enhance conductivity. Device 10 may generally conform to TUNA devices commercially available from Medtronic, Inc, of Minneapolis, Minn. Device 10 further includes, however, fluid delivery conduits and ports for delivery of the fluid, as well as other features that will be apparent from this description.

As shown in FIG. 1, device 10 includes a manipulator 12 having a handle 14, a barrel 16, and a catheter 18 extending from the barrel. A trigger-like actuator 20 is actuated to advance an electrically conductive ablation needle 19 from a distal end 21 of catheter 18. device 10 further includes an endoscope viewfinder 22 coupled to an endoscopic imaging device that extends along the length of catheter 18.

A fluid delivery port 24 is coupled to a fluid delivery lumen (not shown) that extends along the length of catheter 18 to deliver fluid to distal end 21. A proximal end of fluid delivery port 24 is coupled to a fluid delivery device 26 that includes a reservoir containing a fluid and hardware to transmit the fluid to fluid delivery port 24. For example, fluid delivery device 26 may include a pump, a syringe, or other mechanisms to transmit the fluid under pressure.

An ablation current cable 28 is coupled to an electrical conductor that extends along the length of catheter 18 to needle 19. A proximal end of cable 28 is coupled to an ablation energy generator 30 via an electrical connector 32. Ablation energy generator 30 is also coupled to a reference electrode 34, which may be placed on or within the patient to complete an electrical circuit for transmission of current to the target tissue site.

In operation, a surgeon introduces catheter 18 into urethra 36 of a male patient, and advances the catheter so that distal end 21 is deployed adjacent the prostate. Endoscopic viewfinder 22 may aid in positioning distal end 21 of catheter 18 relative to the prostates. In particular, distal end 21 is deployed between lateral lobes 42, 44 in the example of FIG. 1. Needle is extended from distal end 21 of catheter 18 to penetrate the urethral wall and one of the prostate lobes 42, 44. In some embodiments, catheter 18 may carry multiple ablation needles on opposite sides of the catheter to simultaneously access both lobes 42, 44.

Upon penetration of needle 19 into prostate lobe 42, the needle delivers ablation energy from ablation energy generator 30 to ablate tissue within the prostate lobe. Prior to activation of ablation energy generator 30 to deliver ablation current to needle 19, however, fluid delivery device 26 may be activated to deliver the fluid to the target tissue site proximate prostate 42. For example, fluid delivery device 26 may deliver a fluid that is loaded with an anesthetic agent as well as conductive material. In this manner, the fluid serves the dual purpose of relieving pain induced by the ablation procedure and creating a virtual electrode to enhance the ablation procedure.

Needle 19 or distal end 21 of catheter 21, or both, may include one or more ports for emission of the fluid. The fluid may be sufficiently viscous to provide a controllable flow within catheter 18 and out of distal end 21 of catheter 18. Fluid delivery device 26 may be activated to deliver the fluid before, during and after the ablation procedure. For example, the material may be delivered before the ablation needle 19 is activated in order to prepare the tissue in and around prostate gland 42 for delivery of the ablation energy.

For example, delivery of the fluid prior to ablation establishes the virtual electrode shape and volume, and allows the anesthetic agent a short period of time to take effect. In addition, catheter 18 may continue to deliver the fluid during the course of the ablation procedure to replenish material that may be consumed by the ablation energy, and to continue to deliver the anesthetic agent to maintain pain relief. In addition, the fluid may be delivered after the ablation energy is deactivated, providing continued pain relief for a short period of time before needle 19 is withdrawn from prostate 42. In some embodiments, the concentration of anesthetic agents and conductive material may be modulated in stages so that different concentrations are delivered within the stages prior to, during and after ablation.

The fluid may be transmitted to the target tissue site, i.e., the region adjacent prostate lobes 42, 44, by a fluid delivery lumen coupled to needle 19. In particular, needle 19 may be hollow and include one or more fluid delivery ports, as will be described. Hence, the fluid may be delivered via the same needle 19 used to deliver ablation energy to prostate lobe 42. Alternatively, the fluid may be emitted from one or more ports formed in the body of catheter 18 adjacent distal end 21.

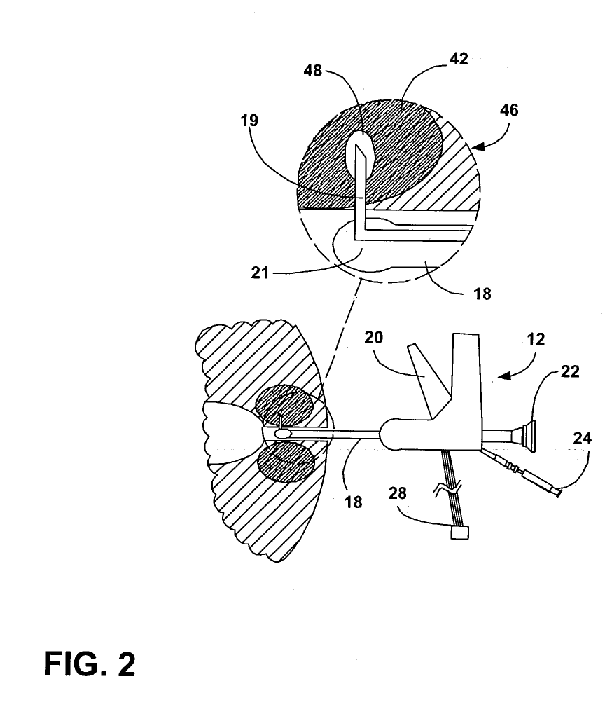

FIG. 2 is an enlarged view of the distal end 21 of device 10 of FIG. 1. As shown in enlarged region 46, distal end 21 of catheter 18 includes an aperture that permits needle 19 to extend outward from the catheter to penetrate lateral prostate lobe 42. Upon application of the fluid via needle 19, prostate tissue within lobe 42 receives an anesthetic agent that prepares the tissue in order to reduce pain associated with ablation. The fluid creates a volume 48 within prostate 42. Upon application of ablation current, needle 19 creates a zone of ablated tissue within volume 48. Propagation of ablation current and effective ablation of prostate tissue are aided by the conductive material dispersed in volume 48 of the fluid.

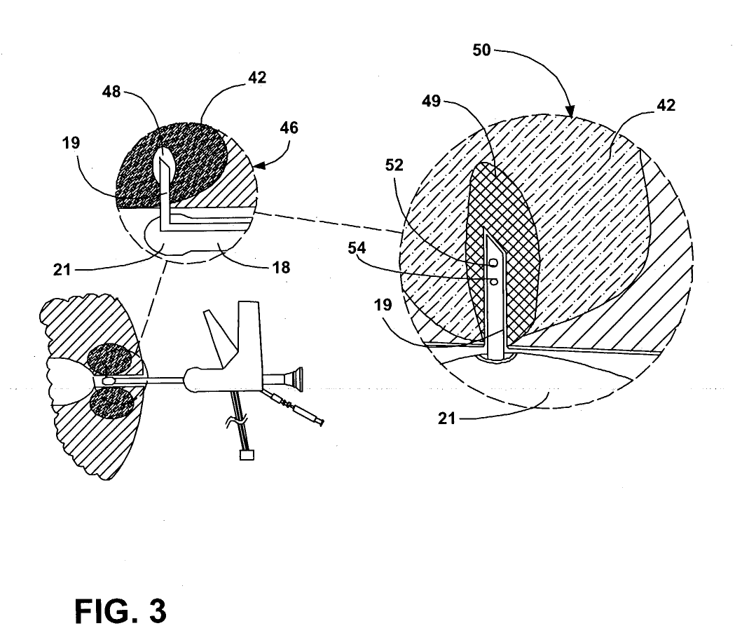

FIG. 3 is another enlarged view of the distal end of the device of FIG. 1. As shown in enlarged region 50, needle 19 may include fluid delivery ports 52, 54 for delivery of the fluid into the tissue of prostate lobe 42. Needle 19 may be constructed of a highly flexible, conductive metal such as nickel-titanium alloy, tempered steel, stainless steel, beryllium-copper alloy and the like. Nickel-titanium and similar highly flexible, shaped memory alloys are preferred. Needle 19 defines an internal lumen (not shown) in fluid communication with fluid delivery ports 52, 54.

In general, the electrical ablation current delivered by needle 40 may be selected to provide pulsed or sinusoidal waveforms, cutting waves, or blended waveforms that are effective in killing cells within the tissue site. In addition, the electrical current may include ablation current followed by current sufficient to cauterize blood vessels. The electrical current is accompanied by delivery of the fluid, which is loaded with conductive parties to yield desired conduction characteristics.

The characteristics of the electrical ablation current are selected to achieve significant cell destruction within the target tissue site. The electrical ablation current may comprise radio frequency (RF) current in the range of approximately 5 to 300 watts, and more preferably 5 to 50 watts, and can be applied for a duration of approximately 15 seconds to 3 minutes. If electrocautery is also provided via needle 10, then ablation energy generator 30 also may generate electrocautery waveforms. Electrical ablation current flows between ablation needle 19 and a reference electrode 34 placed within or on the surface of the patient's body. Alternatively, ablation needle 19 may take the form of a bipolar probe that carries two or more ablation electrodes, in which case the current flows between the electrodes.

As shown in FIG. 3, the ablation current delivered via needle 19 and the virtual electrode created by the fluid creates a larger and more precise lesion 49 within prostate tissue 42. At the same time, the anesthetic agent in the fluid alleviates the pain caused by the creation of the lesion, however, and makes the patient more comfortable during the procedure. In particular, the anesthetic agent can relieve the burning/heating sensation reported by many transurethral ablation patients during the first thirty seconds of delivery of the ablation current, making the patient more comfortable.

The fluid may be delivered with syringe pressure or higher pressures to potentially penetrate the tissue. In some embodiments, the fluid may be intentionally transmitted at a pressure sufficient to destroy or damage some of the tissue surrounding needle 19A. The fluid can be injected at one central position or multiple positions within a respective prostate lobe. For example, it may be useful to penetrate the prostate lobe at multiple positions, e.g., at the 3 and 9 o'clock positions in the mid-portion of the lobe. Exemplary locations for injection of anesthetic agents are described, for example, in George W. Yu, Critical Operative Maneuvers in Urologic Surgery, Chapter 21.1, page 7.

In some embodiments, needle 19 of catheter 18 can deliver a full dosage in all sites first, and then return to the sites for ablation of the lateral and median lobes. Alternatively, with each needle penetration, or “stick,” needle 19 can inject a full dosage of the fluid and then ablate before removing the needle. Also, the fluid may be delivered at an efficacious flow rate before, during and after the ablation.

In addition, a patient may be provided with a control mechanism, such as a thumb switch, to deliver additional amounts of the fluid while the physician performs the lesion. The thumb switch may be coupled to a fluid pump to infuse more pain medication. Additional effects of constant perfusion with the fluid are natural cooling of the needle tip, which can reduce charring and burning at the needle tip, and potentially result in larger lesions or faster lesions.

Further, in some embodiments, needle 19 may deliver electromagnetic energy prior to penetration of the prostate to help the fluid migrate into the prostatic urethra. For example, lidocaine fluid and lidocaine jelly can be inserted into the urethra for 20 minutes prior to the ablation procedure. Ordinarily, the urethral mucosa is a resistant to the numbing effects of anesthetic agents transferring across the mucosal layer. With application of a pulsed current across the bladder mucosa, however, the lidocaine molecules may be forced through the mucosal wall by electroporation effects. This approach may be useful in reducing pain associated with needle penetration.

The fluid may include a variety of liquid, gels, or liquid suspension containing a variety of anesthetic agents and conductive materials. A virtual electrode can be created by the introduction of a conductive fluid, such as isotonic or hypertonic saline or a gel, into or onto the tissue to be ablated. As described in commonly assigned U.S. Pat. No. 6,537,272 to Christopherson et al., the conductive fluid will facilitate the spread of the current density substantially equally throughout the extent of the flow of the conductive fluid, thus creating a virtual electrode substantially equal in extent to the size of the delivered conductive fluid. RF current can then be passed through the virtual electrode into the tissue.

A virtual electrode can be substantially larger in volume than the needle tip electrode typically used in RF interstitial ablation procedures and thus can create a larger lesion than can a dry, needle tip electrode. That is, the virtual electrode spreads or conducts the RF current density outward from the RF current source into or onto a larger volume of tissue than is possible with instruments that rely on the use of a dry electrode. In other words, the creation of the virtual electrode enables the current to flow with reduced resistance or impedance throughout a larger volume of tissue, thus spreading the resistive heating created by the current flow through a larger volume of tissue and thereby creating a larger lesion than could otherwise be created with a dry electrode.

The fluid is loaded with an anesthetic agent. As an example of a suitable anesthetic agent, a gel material loaded with approximately 18 to 20 ml of 1% lidocaine will achieve a desired anesthetic effect when applied to the prostate tissue. Examples of anesthetic agents includes benzocaine, dyclonine, markaine, sensorcaine, lidocaine, and lidocaine hydrochloride gel, or mixtures thereof. Other possible anesthetic agents are Benzocaine, Butamben, Tetracaine, Dibucaine, Dyclonine, Lidocaine, and Pramoxine or mixtures thereof. In some embodiments, it may be desirable to include a vasoconstrictor to keep the anesthetic effect localized. The prostate is highly vascularized and highly nervated. One problem could be that the anesthetic effect may be limited due to the highly nervated prostate and relatively localized area of delivery. With excellent vascularization, is very likely for anesthetic transference across the prostate via the highly vascularized perfusion system of the prostate.

The fluid may take the form of a biocompatible hydrogel loaded with conductive materials, such as any of a variety of biocompatible, conductive salts, and anesthetic agents as described above, The conductive salts serve to conduct RF electrical current throughout the volume of the fluid applied to the prostate, thereby increasing the effective volume of the lesion created by application of ablation current.

The fluid delivered via the transurethral ablation catheter 18 may include a steroid to promote healing of prostate tissue following the ablation procedure. The steroid may be mixed with the conductive fluid, and may be mixed with the anesthetic agent in the fluid. In this case, the steroid may be delivered before, during or after the ablation procedure. Alternatively, the steroid may be delivered independently of the conductive/anesthetic fluid. For example, the steroid may be delivered following the ablation procedure. In addition, the fluid may carry a vaso-constrictor that serves to constrict blood vessels in the ablation zone. In this manner, the vaso-constrictor tends to reduce blood flow that otherwise would contribute to cooling in the ablation zone, and thereby reduce the concentration of ablation energy and prolong the time needed for effective ablation.

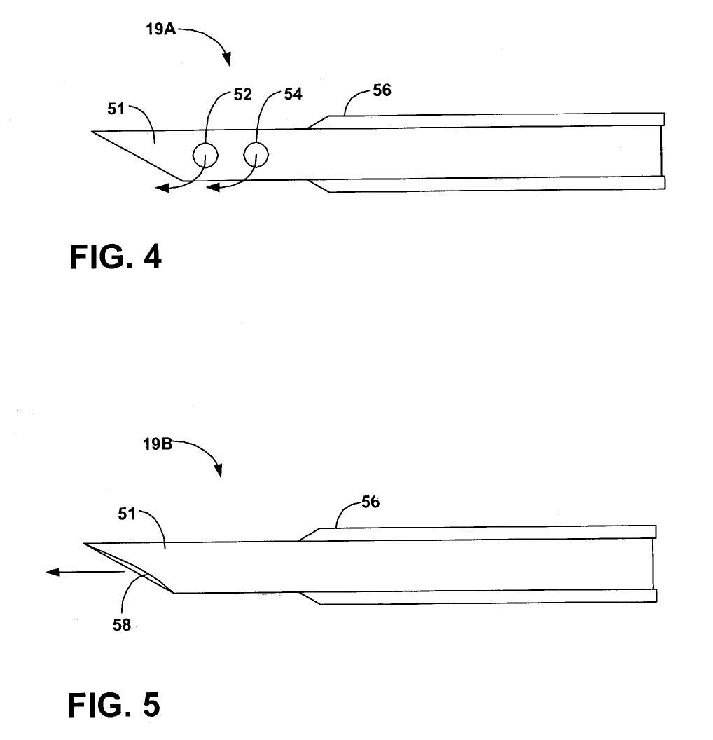

FIG. 4 is a side view of an ablation needle 19A equipped to deliver a fluid to a target tissue site. As shown in FIG. 4, ablation needle 19A may include an insulative layer 56 and a needle body 51. Needle body 51 defines fluid delivery ports 52, 54 for lateral delivery of the fluid into tissue proximate the needle body. The number of fluid delivery ports 52, 54 may vary. In addition, additional fluid delivery ports may be formed at opposite sides of needle body 51, or at different circumferential positions about the periphery of the needle body.

The length of needle 19 may be on the order of approximately 12 to 22 mm. However, needle lengths of up to 50 mm may be desirable to deliver the fluid to the ends of the prostatic capsule. Additional, it may be desirable to perfuse the fluid through the entire 50 mm depth to ensure maximum anesthetic effect, and then withdraw the needle to the 12 to 22 mm needle depth range before ablation to create a lesion is performed.

FIG. 5 is a side view of another ablation needle 19B equipped to deliver a fluid to a target tissue site. In the example of FIG. 5, needle 19B generally conforms to needle 19A. However, needle 19B does not include lateral fluid delivery ports. Instead, needle 19B includes a distal fluid delivery port 58.

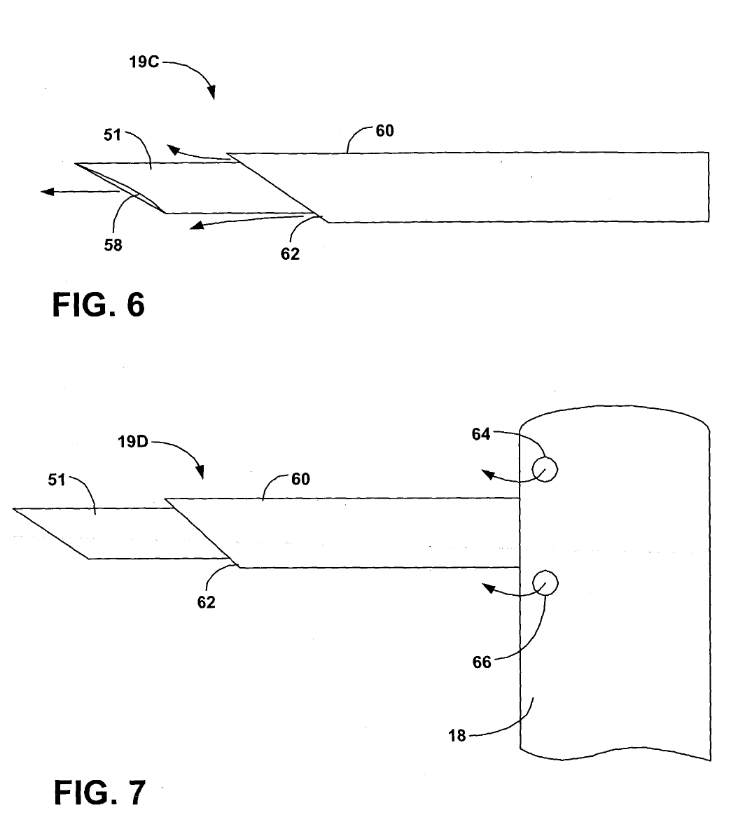

FIG. 6 is a side view of another alternative ablation needle 19C equipped to deliver a fluid to a target tissue site. In the example of FIG. 6, ablation needle 19C includes a concentric tube arrangement comprising needle body 51 and an outer tube 60. The annular space defined between outer tube 60 and needle body 51 forms a fluid delivery port 62. In some embodiments, needle body 51 also may include a distal fluid delivery port 58.

FIG. 7 a side view of a further alternative ablation needle 19C equipped to deliver a fluid to a target tissue site. In the example of FIG. 7, ablation needle 19C extends outward from distal end 21 of catheter 18. Ablation needle 19C may define an annular fluid delivery port 62 between outer tube 60 and needle body 51. In addition, distal end 21 of catheter 18 includes fluid delivery ports 64, 66. Fluid delivery ports 64, 66 permit delivery of the fluid to not only the prostate tissue in which needle 19C is extended, but also the urethral wall during placement of introduction of catheter 18.

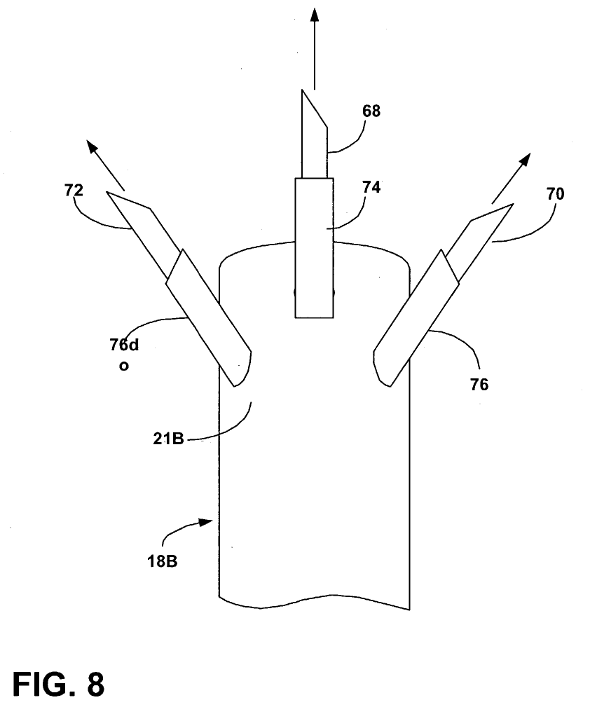

FIG. 8 is a side view of a distal end 21B of an ablation catheter 18B incorporating multiple ablation needles 68, 70, 72 for delivery of ablation current and a fluid. In the example of FIG. 8, ablation needles 68, 70, 72 are mounted at positions appropriate for access to the central, right lateral, and left lateral prostate lobes, respectively. Each needle is may extend from a respective insulative sleeve 74, 76, 78. Ablation needles 68, 70, 72 define respective distal fluid delivery ports for delivery of the fluid.

In operation, using manipulator 12, the surgeon may initially translate and rotate catheter 18, for example, to bring one or more needles 19 into alignment with one of the prostate lobes. If catheter 18 includes only a single needle, the surgeon may rotate the catheter, following ablation of tissue within the desired lobe, to access the other lateral lobe and the medial lobe, if desired. Alternatively, as mentioned above with respect to FIG. 8, catheter 18 may include two or more probes oriented to penetrate the lateral lobes simultaneously. Longitudinal and radial positioning of catheter 18 may be aided by endoscopic viewfinder 22 (FIG. 1), or other imaging techniques such as ultrasound, MRI or the like.

Upon deployment of distal end 21 proximate a target tissue site within the urethra, ablation needle 19 is inserted into the prostate tissue. For example, a surgeon may use actuator 20 (FIG. 1) to drive needle 19 through the urethral wall and into prostate tissue 42. When needle 19 is lodged in the prostate tissue, the surgeon activates fluid delivery device 26 (FIG. 1) to deliver the fluid along the length of catheter 18 to needle 19. Needle 19 emits the fluid into the prostate tissue to provide an anesthetic effect and create a volume of conductive fluid for use as a virtual electrode.

After an amount of time sufficient for the anesthetic agent to take effect, the surgeon activates ablation energy generator 19 to deliver ablation energy to the tissue site via needle 19. Upon application of ablation current, needle 19 ablates a zone of tissue surrounding the needle. The surgeon may continue to delivery the fluid to the target tissue site during the delivery of ablation current. In addition, the surgeon may continue to deliver the fluid following the ablation procedure before withdrawing needle 19 from the target tissue site.

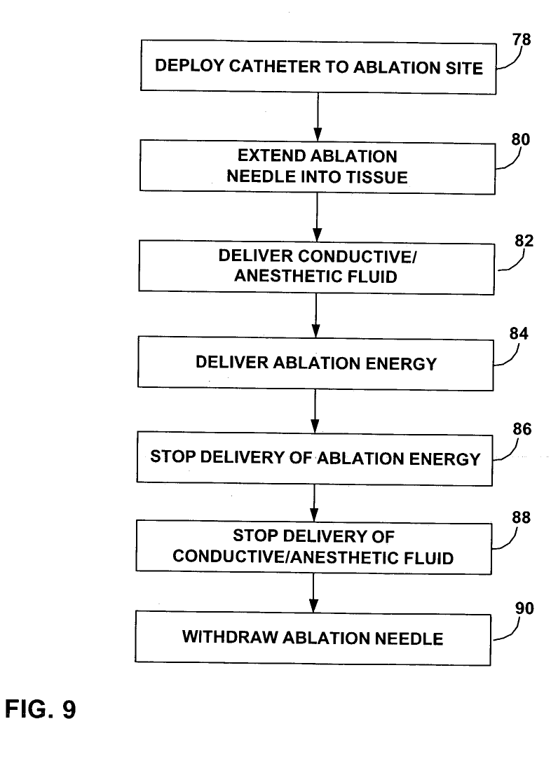

FIG. 9 is a flow diagram illustrating a transurethral ablation procedure. As shown in FIG. 9, the procedure involves deploying a catheter to an ablation site (78), e.g., the prostate reached by transurethral deployment. Upon extension of an ablation needle into the target tissue (80), the fluid is delivered (82) to prevent or alleviate pain associated with the penetration of the urethral wall and prostate tissue, as well as pain associated with delivery of ablation energy. In addition, the fluid may contain conductive material to create a virtual electrode.

Then, following a period of time for the anesthetic agents in the fluid to take effect, ablation energy is applied (84). Much of the pain associated with ablation occurs within the first thirty to sixty seconds of creating a lesion. Therefore, pre-treatment of the prostate tissue may be sufficient to provide significant pain relief during that period of time. However, it may be desirable to continue delivery of the fluid during the ablation procedure. The ablation energy ablate cells within the target tissue site. When delivery of the ablation energy is stopped (86), delivery of the fluid may also be stopped (88). Alternatively, the fluid may continue to be delivered for a period of time following termination of the ablation energy. Then, the ablation needle and catheter are withdrawn from the patient (90).

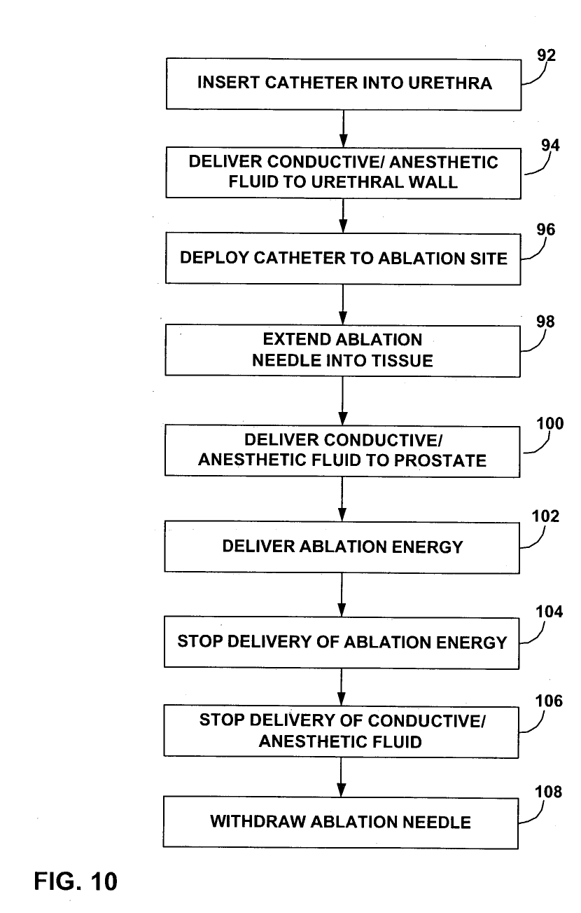

FIG. 10 is a flow diagram illustrating another transurethral ablation procedure. As shown in FIG. 10, the procedure involves inserting a catheter into the urethra (92). In addition, the fluid is delivered upon introduction of the catheter (94). Accordingly, in this embodiment, the catheter body may include fluid delivery ports. In this manner, the catheter delivers fluid to provide an anesthetic agent that prevents pain caused by trauma to the urethral wall. If the fluid also contains a conductive material, care may be taken to avoid undesirable extension of the virtual electrode in a manner that would produce substantial ablation of the urethral wall of other non-prostatic tissue.

Upon deploying the catheter to an ablation site (96), e.g., the prostate, the ablation needle is extended into the target tissue (98), and the fluid is delivered (100) to prevent or alleviate pain associated with the penetration of the urethral wall and prostate tissue, as well as pain associated with delivery of ablation energy, and to create a virtual electrode for more effective volumetric coverage and precision. Ablation energy is then delivered via the probe (102) to ablate cells with the target tissue site. When delivery of the ablation energy is stopped (104), delivery of the fluid may also be stopped (106), or continued for a period of time following termination of the ablation energy. Then, the ablation needle and catheter are withdrawn from the patient (108).

As further features, a controller may be provided to coordinate the timing and duration of delivery of ablation current and the fluid by ablation energy generator 30 and fluid delivery device 26, respectively. For example, the controller may execute a surgeon-programmable routine to selectively activate fluid delivery during the course of ablation.

The invention can provide a number of advantages. In general, the invention may reduce the pain associated with some existing transurethral ablation techniques. Also, the invention offers a localized treatment for alleviation of pain. In addition, in some embodiments, the fluid can be delivered by the same device used to perform the transurethral ablation procedure, making the procedure less complex, quicker, and more convenient for the surgeon. As a further advantage, the invention provides greater volumetric coverage and precision in the ablation procedure, enabling a greater volume of prostate tissue to be more uniformly ablated within a given ablation procedure.

In addition, the invention may permit direct visualization of anesthesia delivery, e.g., through an endoscope provided with transurethral ablation device 10. The invention also eliminates the need for a transperineal prostatic block, sedation or general anesthesia. The most common block is the perineal prostatic block which typically is done under ultrasound guidance. The invention would remove the need to have an ultrasound device to deliver pain medication, and remove the need for additional equipment, e.g., syringe and needle, to deliver the perineal prostatic block. In this manner, the invention simplifies delivery of pain relief along with ablation delivery.

As a further advantage, the virtual electrode formed by fluid delivery supports controlled ablation within a larger, yet more precise, zone of prostate tissue. With continued delivery of fluid during ablation, the efficacy of the lesion either in size, or time to develop lesion size, may be improved. In addition, continued delivery of fluid during ablation may reduce or eliminate the need for fluid delivery to cool the urethra, e.g., by delivering fluid out of the catheter and into the urethra.

In general, the invention can shorten the total ablation procedure time by shortening the amount of time spent sedating the patient and the time otherwise spent in performing the prostatic block. Also, the use of a virtual electrode shortens overall ablation time and directly impacts pain. Another advantage concerns the potential for patient directed delivery of the fluid to alleviate pain on demand. In addition, the invention may support electromotive transfer, e.g., by electroporation, to more effectively drive the anesthetic agent into the tissue.

The preceding specific embodiments are illustrative of the practice of the invention. It is to be understood, therefore, that other expedients known to those skilled in the art or disclosed herein may be employed without departing from the invention or the scope of the claims. For example, the present invention further includes within its scope methods of making and using systems for transurethral ablation, as described herein.

In addition, although the disclosure refers to an ablation needle for purposes of illustration, delivery of fluids carrying anesthetic agents also may be desirable with other types of ablation probes, such as optical waveguides for delivery of laser energy, microwave probes, and cryogenic probes.

In the claims, means-plus-function clauses are intended to cover the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts a nail and a screw are equivalent structures.

Many embodiments of the invention have been described. Various modifications may be made without departing from the scope of the claims. These and other embodiments are within the scope of the following claims.

1. A method comprising:
delivering an electrically conductive fluid containing an anesthetic agent to a prostate of a male patient; and
delivering ablation energy to the prostate.

2. The method of claim 1, further comprising:
inserting a catheter into a urethra of the patient; and
delivering the fluid via an ablation needle carried by the catheter.

3. The method of claim 1, further comprising delivering the fluid via a catheter.

4. The method of claim 1, further comprising delivering the fluid before the delivery of the ablation energy.

5. The method of claim 1, further comprising delivering the fluid during the delivery of the ablation energy.

6. The method of claim 1, further comprising stopping delivery of the ablation energy, and delivering the fluid after stopping delivery of the ablation energy.

7. The method of claim 1, wherein the ablation energy includes electrical current selected to kill cells within the prostate.

8. The method of claim 1, wherein the fluid includes a gel-based material loaded with the anesthetic agent and conductive material.

9. The method of claim 8, wherein the anesthetic agent includes at least one of benzocaine, dyclonine, markaine, sensorcaine, lidocaine, and lidocaine hydrochloride.

10. The method of claim 1, further comprising penetrating a wall of a urethra of the patient with an ablation needle, extending the ablation needle into the prostate, delivering the fluid to the prostate via the ablation needle, and delivering the ablation energy to the prostate via the ablation needle.

11. The method of claim 1, further comprising delivering the fluid to a wall of the urethra of the patient.

12. The method of claim 1, further comprising delivering the fluid via a fluid deliver port in a catheter.

13. The method of claim 1, further comprising delivering the fluid and the ablation energy via a transurethral catheter.

14. The method of claim 1, wherein the fluid is a gel.

15. The method of claim 1, wherein the fluid includes a steroid.

16. The method of claim 1, wherein the fluid includes a vaso-constrictor.

17. A transurethral ablation system comprising:
a transurethral catheter;
an ablation needle extendable from the catheter to penetrate a prostate of a patient;
a fluid delivery conduit extending within the catheter;
an ablation energy generator to deliver ablation energy to the prostate via the ablation probe; and
a fluid delivery device to deliver fluid to the prostate via the fluid delivery conduit.

18. The system of claim 17, further comprising a fluid delivery port formed in the ablation needle and coupled to the fluid delivery conduit.

19. The system of claim 17, further comprising a fluid delivery port formed in the catheter and coupled to the fluid delivery conduit.

20. The system of claim 17, wherein the fluid delivery device delivers an electrically conductive fluid containing an anesthetic agent.

21. The system of claim 17, wherein the ablation needle includes an electrically conductive needle, the ablation energy and the fluid both being delivered to the prostate via the needle.

22. The system of claim 17, further comprising a fluid delivery port formed in the catheter to deliver the fluid to a wall of a urethra of the patient.

23. The system of claim 17, wherein the fluid includes a steroid.

24. The system of claim 17, wherein the fluid includes a vaso-constrictor.
